(19) United States
(12) Patent Application Publication
(10) Pub. No.: US 2012/0301948 A1
(43) Pub. Date: Nov. 29, 2012

(54) Novel method of acid hydrolysis of biomass and the recovery of sugars thereof by solvent extraction

(75) Inventors: Timothy Charles R. Brennan, Brisbane (AU); Bradley M. Holmes, Oakland, CA (US); Blake A. Simmons, San Francisco, CA (US); Harvey W. Blanch, San Francisco, CA (US)

(73) Assignees: SANDIA CORPORATION, Livermore, CA (US); THE REGENTS OF THE UNIVERSITY OF CALIFORNIA, Oakland, CA (US)

(21) Appl. No.: 13/436,704

(22) Filed: Mar. 30, 2012

Related U.S. Application Data
(60) Provisional application No. 61/470,425, filed on Mar. 31, 2011.

(51) Int. Cl. C13K 1/02; C07H 1/06; C12N 1/00; C09K 3/00

(52) U.S. Cl. 435/243; 127/37; 252/182.12; 536/127

(57) ABSTRACT

The present invention provides for a method of hydrolyzing a cellulose, hemicellulose, or ligno-cellulose comprising: (a) contacting (i) an ionic liquid (IL) or ionic liquid-aqueous (ILA) phase comprising cellulose, hemicellulose, or ligno-cellulose, or a mixture thereof, and (ii) an acid, such that the cellulose, hemicellulose, or ligno-cellulose is hydrolyzed into sugar, and (b) optionally adding water to the IL or ILA phase wherein the proportion of water in the IL or ILA phase does not exceed about 60% by weight. The present invention also provides for a method of recovering a sugar comprising contacting an IL or ILA phase and an organic phase comprising an organic acid.

### CROSS-REFERENCE TO RELATED APPLICATIONS

This application claims priority to U.S. Provisional Patent Application Ser. No. 61/470,425, filed Mar. 31, 2011; which is hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of saccharification of biomass using ionic liquid.

### BACKGROUND OF THE INVENTION

The most promising route for large-scale adoption of biofuels as an alternative fuel source is manufacturing simple, fermentable sugars from environmentally sustainable feedstocks, such as agricultural residues, woody biomass, and dedicated energy crops such as switchgrass and Miscanthus. These feedstocks are then converted into monomeric sugars and fermented into biofuel.

Unlike the sugars from starch-based fuel crops (such as those derived from corn and sugarcane), it is much more difficult to produce glucose from the woods and grasses. Lignocellulosic biomass is composed of cellulose, hemicellulose, and lignin that have evolved to be resistant to breakdown into their components. Many different techniques have been attempted for the cost and energy-effective breaking down of cellulose and hemicellulose into monomeric sugars. The enzymatic conversion of biomass to its component sugars is predicated on a pretreatment step that breaks apart the lignin-carbohydrate complex and increases the surface accessibility of the polysaccharides to the hydrolytic enzymes. One of the most critical needs that must be addressed in order for lignocellulosic biofuels to become a reality is a cost-effective and efficient biomass-pretreatment technology that can generate cheap sugar. It is estimated that, on a per-gallon basis, biomass pretreatment represents the second-largest cost in biofuel production for the entire supply chain, after the feedstock. The enzymes used to liberate monomeric sugars from the pretreated biomass also represent a significant cost, estimated to be between $0.30-1.20/gallon of fuel produced.

Several physical and chemical pretreatment methods, typically technologies that have been derived from the pulp and paper industry, are being further developed to overcome the recalcitrance of lignocellulose, increase enzyme efficiency, and improve the yields of monomeric sugars from lignocellulose. These include dilute acid, ammonia fiber expansion, lime, steam explosion, and organic solvent pretreatment method. We have developed a new process that can break down lignocellulose into its constituent sugars—pentose (five-carbon, [5C] sugar) and hexose (six-carbon [6C] sugar)—without the need for enzymes. The sugars produced are selectively extracted into an output stream that is suitable for the production of biofuels.

The production of cheap sugars from biomass is essential to the commercialization of lignocellulosic biofuels. One route to the production of sugars is the chemical hydrolysis of biomass, using acids or other reagents or catalysts. In aqueous solution, acids are limited by their efficacy due to the insolubility of biomass in water. Several room temperature ionic liquids, defined as salts that are in a liquid state below 100° C., are capable of dissolving biomass. By performing the chemical hydrolysis of biomass dissolved in ionic liquid, it is possible to rapidly accelerate the hydrolysis of cellulose and hemicellulose into their component sugars. (Binder and Raines, “Fermentable sugars by chemical hydrolysis of biomass”, Proc. Natl. Acad. Sci., 2010).

While hydrolysis is extremely efficient, the process is hampered by the need to separate the sugars, which are water soluble, from the ionic liquid, which is also water soluble. Previously, chromatography had been proposed as a solution to this problem, but it is both and expensive unit operation, and requires dilution of the highly viscous ionic liquid, which must subsequently be removed.

### SUMMARY OF THE INVENTION

The present invention provides for a method of hydrolyzing a cellulose, hemicellulose, or ligno-cellulose comprising: (a) contacting (i) an ionic liquid (IL) or ionic liquid-aqueous (ILA) phase comprising cellulose, hemicellulose, or ligno-cellulose, or a mixture thereof, and (ii) an acid, such that the cellulose, hemicellulose, or ligno-cellulose is hydrolyzed into sugar, and (b) optionally adding water to the IL or ILA phase wherein the proportion of water in the IL or ILA phase does not exceed about 60% by weight. In some embodiments of the invention, the acid has a pH equal to or lower than about the pH of 4M hydrochloric acid. In some embodiments of the invention, the acid is an inorganic acid, such as HCl, sulfuric acid, or nitric acid.

In some embodiments of the invention, the method of hydrolyzing a cellulose, hemicellulose, or ligno-cellulose can further comprise (c) contacting the ionic liquid (IL) or ionic liquid-aqueous (ILA) phase and an organic phase optionally comprising an organic acid, wherein the solution comprises an ionic liquid, a sugar and a boronic acid; (d) contacting the sugar with the boronic acid to form a sugar-boronic acid complex, (e) separating the organic phase and the aqueous phase, wherein the organic phase contains the sugar-boronic acid complex, (f) optionally adding a stripping agent to the organic phase and optionally (g) separating the sugar from the organic phase.

In some embodiments of the invention, the method of hydrolyzing a cellulose, hemicellulose, or ligno-cellulose can further comprise one or more steps of removing a sugar from a solution described in U.S. Provisional Patent Application Ser. No. 61/246,911, filed Sep. 29, 2009, PCT International Patent Application No. PCT/US2010/050778, filed Sep. 29, 2010, and U.S. patent application Ser. No. 13/434,714, filed Mar. 29, 2012 (all of which are hereby incorporated by reference).

In one embodiment of the invention, the method preferentially extracts sugars produced by the direct acid hydrolysis of real biomass, from an aqueous solution of ionic liquids using liquid liquid extraction. The method comprises dissolving biomass in an ionic liquid, such as 1-ethyl-3-methylimidazolium chloride at from about 105° C. to about 140° C. for 6 hours. The temperature is then reduced to 105° C. and an aqueous mixture of HCl is added. Additional water is titrated into the reaction over time to minimize the formation of sugar dehydration products by the acid. Once the concentration of water is ˜40 wt %, the sugars are extracted into an organic phase comprising napthalene-2-boronic acid, hexane and the phase transfer catalyst aliquat 336. Extraction of upwards of 80% of the hexose and pentose sugars can be achieved.

The present invention also provides for a composition comprising a solution comprising (a) an ionic liquid (IL) or ionic liquid-aqueous (ILA) phase and (b) an organic phase, wherein the solution comprises a sugar, a boronic acid, and an organic acid. The boronic acid can be of a trigonal form or a tetrahedral anion form. In some embodiments of the invention, the sugar and the boronic acid form a sugar-boronic acid complex which is in the organic phase. The boronic acid in the sugar-boronic acid complex is in the tetrahedral anion form. In some embodiments of the invention, the sugar is derived from a biomass feedstock. In some embodiments of the invention, the sugar is derived from the hydrolysis of cellulose, hemicellulose, or ligno-cellulose, or a mixture thereof. In some embodiments of the invention, the organic acid is a carboxylic acid, such as an alkanoic acid, such as hexanoic acid.

The present invention provides for a method of removing a sugar from a solution, comprising: (a) providing a solution comprising (i) an ionic liquid (IL) or ionic liquid-aqueous (ILA) phase and (ii) an organic phase comprising an organic acid, wherein the solution comprises an ionic liquid, a sugar and a boronic acid; (b) contacting the sugar with the boronic acid to form a sugar-boronic acid complex, (c) separating the organic phase and the aqueous phase, wherein the organic phase contains the sugar-boronic acid complex, and optionally (d) separating the sugar from the organic phase.

In some embodiments of the invention, the (a) providing step comprises contacting (i) an IL or ILA solution and (ii) an organic solution comprising an organic acid. In some embodiments of the invention, the IL or ILA solution comprises ionic liquid (IL), a sugar, and a boronic acid.

In some embodiments of the invention, the (d) separating step comprises the sugar-boronic acid complex dissociating and the sugar moving into a second IL, ILA, or aqueous solution. In some embodiments of the invention, the (d) separating step further comprises separating the second IL, ILA, or aqueous solution from the organic phase.

In some embodiments of the invention, the method further comprises culturing a cell using the sugar obtained from the (d) separating step. In some embodiments of the invention, the cell produces a molecule of interest. In some embodiments of the invention, the molecule of interest is a biofuel.

In some embodiments of the invention, the biomass is a cellulose biomass, hemicellulose biomass, ligno-cellulose biomass, or a mixture thereof.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows the acidolysis of switchgrass at 105° C. using 10 wt % (to biomass) HCl for 2.5 hrs. The dissolution of switchgrass in [C2mim][Cl] was for 5 hrs at the specified temperature and then allowed to equilibrate to the reaction temperature of 105° C. for the last hour. Total dissolution time was 6 hr. Sugar yields are molar yields analyzed using HPAEC and based on sugar units contained in the switchgrass feedstock.

FIG. 2 shows the extraction of glucose from the acidolysis-IL supernatant with an organic phase containing 70 mM naphthalene-2-boronic acid in hexane for 2 hrs at 70° C. Sodium hydroxide (NaOH) was added using varying amounts of 10M NaOH. The final reaction volume was not affected and the addition of NaOH can be treated as negligible.

FIG. 3 shows the acidolysis of swithgrass in [C2mim][Cl] at 105° C. for 2.5 hr. The amount of HCl was varied (wt % to biomass) keeping the [C2mim][Cl] weight, switchgrass (5 wt %) and water addition constant. Sugar yields are molar yields analyzed using HPAEC and based on sugar units contained in the switchgrass feedstock.

FIG. 4 shows the boronic acid-ester sugar complex and proposed mechanism for sugar release.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “IL” includes a single IL compound as well as a plurality of IL compounds, either the same (e.g., the same molecule) or different.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

When the term “boronic acid” is used, it includes the boronate form.

The present invention also provides for a composition comprising a solution comprising (a) an ionic liquid (IL) or ionic liquid-aqueous (ILA) phase and (b) an organic phase, wherein the solution comprises a sugar, a boronic acid, and an organic acid. The boronic acid can be of a trigonal form or a tetrahedral anion form. In some embodiments of the invention, the sugar and the boronic acid form a sugar-boronic acid complex which is in the organic phase. The boronic acid in the sugar-boronic acid complex is in the tetrahedral anion form. In some embodiments of the invention, the sugar is derived from a biomass feedstock. In some embodiments of the invention, the sugar is derived from the hydrolysis of cellulose, hemicellulose, or ligno-cellulose, or a mixture thereof. In some embodiments of the invention, the organic acid is a carboxylic acid, such as an alkanoic acid. In some embodiments of the invention, the alkanoic acid comprises equal to or less than about 10 carbon atoms, such as hexanoic acid.

The sugars that can be used in this present invention are any sugar that comprises a cis-diol, such as including, but are not limited to, monosaccharides, such as 6-carbon monosaccharides (such as glucose) and 5-carbon monosaccharides (such as xylose), disaccharides, and other complex sugars, such as cellobiose. In some embodiments of the invention, the sugar is derived from a biomass feedstock. In some embodiments of the invention, when the sugar is derived from the method of hydrolyzing a cellulose, hemicellulose, or ligno-cellulose, or acidolysis as described herein, then the use of a cellulase to hydrolyze the biomass or cellulose is not required. In some embodiments of the invention, the sugar is derived from the hydrolysis of cellulose. The hydrolysis of cellulose can be by any suitable cellulase, such as an endoglucanase and/or an exoglucanase. In some embodiments of the invention, the suitable cellulase is a thermostable or thermophilic cellulase, or a mutant thereof. Such suitable cellulases, or mutants thereof, are taught in U.S. Provisional Patent Application Ser. Nos. 61/172,653, 61/172,668, and 61/246,439; U.S. patent application Ser. No. 12/892,724; and, PCT International Patent Application No. PCT/US2010/032320 (all of which are hereby incorporated by reference).

In some embodiments of the invention, the IL or ILA phase has a pH of more than about 9. The pH of the IL or ILA phase has to be higher than the pKa of the boronic acid in order for the boronic acid in the IL or ILA phase to form the tetrahedral anion form. In some embodiments, the pH of the IL or ILA phase is equal to or greater than 10, 11, or 12.

### Organic Phase

The organic phase comprises any suitable organic molecule, or a mixture thereof, that is immiscible with the IL and ILA solutions, and the organic molecules do not react with the boronic acid and the sugar. A suitable molecule is Aliquat® 336 (commercially available from Cognis Corp., Cincinnati, Ohio), which is a water insoluble quaternary ammonium salt made by the methylation of mixed tri octy/decyl amine

### Ionic Liquid (IL)

The suitable IL that can be used in the present invention can be any IL that does not impede the forming of the sugar-boronic acid complex. In some embodiments of the invention, the IL is suitable for pretreatment of biomass and for the hydrolysis of cellulose by thermostable cellulase. Suitable IL are taught in ChemFiles (2006) 6(9) (which are commercially available from Sigma-Aldrich; Milwaukee, Wis.). Such suitable IL include, 1-alkyl-3-alkylimidazolium alkanate, 1-alkyl-3-alkylimidazolium alkylsulfate, 1-alkyl-3-alkylimidazolium methylsulfonate, 1-alkyl-3-alkylimidazolium hydrogensulfate, 1-alkyl-3-alkylimidazolium thiocyanate, and 1-alkyl-3-alkylimidazolium halide, wherein an “alkyl” is an alkyl group comprising from 1 to 10 carbon atoms, and an “alkanate” is an alkanate comprising from 1 to 10 carbon atoms. In some embodiments, the “alkyl” is an alkyl group comprising from 1 to 4 carbon atoms. In some embodiments, the “alkyl” is a methyl group, ethyl group or butyl group. In some embodiments, the “alkanate” is an alkanate comprising from 1 to 4 carbon atoms. In some embodiments, the “alkanate” is an acetate. In some embodiments, the halide is chloride.

Such suitable IL include, but are limited to, 1-ethyl-3-methylimidazolium acetate (EMIN Acetate), 1-ethyl-3-methylimidazolium chloride (EMIN Cl), 1-ethyl-3-methylimidazolium hydrogensulfate (EMIM HOSO₃), 1-ethyl-3-methylimidazolium methylsulfate (EMIM MeOSO₃), 1-ethyl-3-methylimidazolium ethylsulfate (EMIM EtOSO₃), 1-ethyl-3-methylimidazolium methanesulfonate (EMIM MeSO₃), 1-ethyl-3-methylimidazolium tetrachloroaluminate (EMIM AlCl₄), 1-ethyl-3-methylimidazolium thiocyanate (EMIM SCN), 1-butyl-3-methylimidazolium acetate (BMIM Acetate), 1-butyl-3-methylimidazolium chloride (BMIM Cl), 1-butyl-3-methylimidazolium hydrogensulfate (BMIM HOSO₃), 1-butyl-3-methylimidazolium methanesulfonate (BMIM MeSO₃), 1-butyl-3-methylimidazolium methylsulfate (BMIM MeOSO₃), 1-butyl-3-methylimidazolium tetrachloroaluminate (BMIM AlCl₄), 1-butyl-3-methylimidazolium thiocyanate (BMIM SCN), 1-ethyl-2,3-dimethylimidazolium ethylsulfate (EDIM EtOSO₃), Tris(2-hydroxyethyl)methylammonium methylsulfate (MTEOA MeOSO₃), 1-methylimidazolium chloride (MIM Cl), 1-methylimidazolium hydrogensulfate (MIM HOSO₃), 1,2,4-trimethylpyrazolium methylsulfate, tributylmethylammonium methylsulfate, choline acetate, choline salicylate, and the like. The ionic liquid can comprises one or a mixture of the compounds. Further ILs are taught in U.S. Pat. No. 6,177,575; hereby incorporated by reference.

The ionic liquid (IL) is of a concentration of more than 0% to 100% of the composition or solution. In some embodiments, the IL is of a concentration of more than 0% to less than 60% of the composition or solution. In some embodiments, the concentration of IL is equal to or more than 1%, equal to or more than 2%, equal to or more than 3%, equal to or more than 5%, equal to or more than 10%, equal to or more than 15%, or equal to or more than 20%. The upper range of the concentration of IL is less than 60%, or equal to or less than 55%.

In some embodiments of the invention, the IL has a concentration from more than 0% to about 50%. In some embodiments of the invention, the IL has a concentration from more than 0% to about 35%. In some embodiments of the invention, the IL has a concentration from more than 0% to about 20%. In some embodiments of the invention, the IL has a concentration from about 5% to about 20%.

The solution containing IL can further comprise NaCl, such as up to 10 mM of NaCl. The solution can further comprise a suitable buffer.

### Boronic Acid

Suitable boronic acids that can be used in the present invention are boronic acids that can form a tetrahedral anion form which can in turn complex with a sugar (see FIG. 10). Such suitable boronic acids include, but are not limited to, boronic acids having the formula: R-α-B(OH)₂ (I), wherein a is a bond or an alkyl or alkenyl chain of 1-10 carbons, R comprises at least 1 aromatic ring, wherein optionally at least one ring is substituted by one or more alkyl groups comprising 1-10 carbons. In some embodiments, α is a bond or an alkyl or alkenyl chain of 1-4 carbons. In some embodiments, α is a bond or an alkyl or alkenyl chain of 1-2 carbons. In some embodiments, α is a —C═C—. In some embodiments, R comprises 1, 2, or 3 aromatic rings. In some embodiments, R is a benzene, optionally comprising 1 or 2 methyl groups. In some embodiments, R is a naphthalene.

Such suitable boronic acids include, but are not limited to, phenylboronic acid, 3,5-dimethylphenylboronic acid, 4-tert-butylphenylboronic acid, trans-β-styreneboronic acid, and naphthalene-2-boronic acid.

### METHODS OF THE PRESENT INVENTION

The present invention provides for a method of hydrolyzing a cellulose, hemicellulose, or ligno-cellulose comprising: (a) contacting (i) an ionic liquid (IL) or ionic liquid-aqueous (ILA) phase comprising cellulose, hemicellulose, or ligno-cellulose, or a mixture thereof, and (ii) an acid, such that the cellulose, hemicellulose, or ligno-cellulose is hydrolyzed into sugar, and (b) optionally adding water to the IL or ILA phase wherein the proportion of water in the IL or ILA phase does not exceed about 60% by weight. In some embodiments of the invention, the (b) adding step comprises adding water to the IL or ILA phase wherein the proportion of water in the IL or ILA phase does not exceed about 50% by weight. In some embodiments of the invention, the (b) adding step comprises adding water to the IL or ILA phase wherein the proportion of water in the IL or ILA phase does not exceed about 40% by weight. The amount of water added is sufficient to prevent the acid from degrading the sugar.

In some embodiments of the invention, the acid has a pH equal to or lower than about the pH of 4M hydrochloric acid. In some embodiments of the invention, the acid is an inorganic acid, such as HCl, sulfuric acid, or nitric acid.

In some embodiments of the invention, the (a) contacting step can further comprise: heating the mixture of (i) and (ii), such heating the mixture to a temperature equal to or less than about 200° C., equal to or less than about 150° C., or equal to or less than about 105° C. In some embodiments of the invention, the (a) contacting step can further comprise: maintaining the mixture at the elevated temperature for equal to or less than about 9 hours, equal to or less than about 6 hours, or equal to or less than about 4.5 hours. In some embodiments of the invention, the (a) contacting step can further comprise: maintaining the mixture at the elevated temperature for equal to or more than about 3 hours, equal to or more than about 4.5 hours, or equal to or more than about 6 hours. The temperature and maintenance should be sufficient to have a substantial yield of sugars from the cellulose, hemicellulose, or ligno-cellulose, or biomass.

In some embodiments of the invention, the yield of the sugars from the hydrolysis method is equal to or more than about the acidolysis yields described in Table 2 herein.

In some embodiments of the invention, the method of hydrolyzing a cellulose, hemicellulose, or ligno-cellulose can further comprise (c) contacting the ionic liquid (IL) or ionic liquid-aqueous (ILA) phase and an organic phase optionally comprising an organic acid, wherein the solution comprises an ionic liquid, a sugar and a boronic acid; (d) contacting the sugar with the boronic acid to form a sugar-boronic acid complex, (e) separating the organic phase and the aqueous phase, wherein the organic phase contains the sugar-boronic acid complex, (f) optionally adding a stripping agent to the organic phase and optionally (g) separating the sugar from the organic phase.

Suitable stripping agents include organic acids which decrease the pH of the solution.

In some embodiments of the invention, the method of hydrolyzing a cellulose, hemicellulose, or ligno-cellulose can further comprise one or more steps of removing a sugar from a solution described in U.S. Provisional Patent Application Ser. No. 61/246,911, filed Sep. 29, 2009, and PCT International Patent Application No. PCT/US2010/050778, filed Sep. 29, 2010 (all of which are hereby incorporated by reference).

In one embodiment of the invention, the method preferentially extracts sugars produced by the direct acid hydrolysis of real biomass, from an aqueous solution of ionic liquids using liquid liquid extraction. The method comprises dissolving biomass in an ionic liquid, such as 1-ethyl-3-methylimidazolium chloride at from about 105° C. to about 140° C. for 6 hours. The temperature is then reduced to 105° C. and an aqueous mixture of HCl is added. Additional water is titrated into the reaction over time to minimize the formation of sugar dehydration products by the acid. Once the concentration of water is ˜40 wt %, the sugars are extracted into an organic phase comprising napthalene-2-boronic acid, hexane and the phase transfer catalyst aliquat 336. Extraction of upwards of 80% of the hexose and pentose sugars can be achieved.

The present invention provides for a method of removing a sugar from a solution, comprising: (a) providing a solution comprising (i) an ionic liquid (IL) or ionic liquid-aqueous (ILA) phase and (ii) an organic phase comprising an organic acid, wherein the solution comprises an ionic liquid, a sugar and a boronic acid; (b) contacting the sugar with the boronic acid to form a sugar-boronic acid complex, (c) separating the organic phase and the aqueous phase, wherein the organic phase contains the sugar-boronic acid complex, and optionally (d) separating the sugar from the organic phase.

In some embodiments of the invention, the (a) providing step comprises contacting (i) an IL or ILA solution and (ii) an organic solution comprising an organic acid. In some embodiments of the invention, the IL or ILA solution comprises ionic liquid (IL), a sugar, and a boronic acid.

In some embodiments of the invention, the (d) separating step comprises the sugar-boronic acid complex dissociating and the sugar moving into the second IL, ILA, or aqueous solution. In some embodiments of the invention, the (d) separating step further comprises separating the second IL, ILA, or aqueous solution from the organic phase.

In some embodiments of the invention, the providing step comprises contacting (i) an IL or ILA solution and an organic solution. In some embodiments of the invention, the IL or ILA solution comprises an ionic liquid (IL) and the sugar. In some embodiments of the invention, the organic solution comprises the boronic acid. In some embodiments of the invention, the organic solution further comprises organic solvents which ensure the boronic acid is fully dissolved in the organic phase, such as n-hexane and 1-octanol, or a mixture thereof (such as n-hexane/1-octanol, 85:5, v/v).

In some embodiments of the invention, the contacting step comprises having the sugar and the boronic acid in the solution to essentially come to equilibrium. In some embodiments of the invention, the contacting step comprises increasing the contact between the sugar in the IL or ILA phase and the boronic acid in the organic phase, such as shaking, agitating, swirling, mixing, or any mechanical means of increasing the surface area of the interface between the IL or ILA phase and the organic phase. Sugars that complex with the boronic acid move into the organic phase.

In some embodiments of the invention, the separating step comprises adding a second IL, ILA, or aqueous solution comprising a stripping agent to the organic solution, such that the sugar-boronic acid complex dissociates and the sugar moves into the second IL, ILA, or aqueous solution. In some embodiments of the invention, the separating step further comprises separating the second IL, ILA, or aqueous solution from the organic phase.

In some embodiments of the invention, the separating step comprises increasing the contact between the second IL, ILA, or aqueous phase and the organic phase, such as shaking, agitating, swirling, mixing, or any mechanical means of increasing the surface area of the interface between the second IL, ILA, or aqueous phase and the organic phase. Sugars that disassociate from the sugar-boronic acid complex move into the IL, ILA, or aqueous phase.

In some embodiments of the invention, the yield of the extraction of sugars is equal to or more than about the extraction yields described in Table 2 herein.

In some embodiments of the invention, the method further comprises culturing a cell using the sugar obtained from the separating step. In some embodiments of the invention, the cell produces a molecule of interest. In some embodiments of the invention, the molecule of interest is a biofuel.

In some embodiments of the invention, the biomass is a cellulose biomass, hemicellulose biomass, ligno-cellulose biomass, or a mixture thereof. The biomass can be an untreated or treated biomass.

### APPLICATIONS

The present invention can be used in the extraction of sugars from biomass. The sugars can be used in all process that use 6-carbon sugars, such as glucose, and the resulting sugars can be used for any intended purpose. The process is of significant interest in biomass processing or biofuels and other biomaterials, paper recycling and pulp processing for paper manufacturing.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Acid Catalysis of Corn Stover

Corn stover (26.7 mg, 54 mmol glucose units, 44 μmol xylose units) and EMIM Cl (502 mg) are mixed at 105° C. for 6 h. To this mixture is added aqueous HCl (1.66 M, 29 μL, equivalent to 5 mg conc. HCl), and the reaction mixture is stirred vigorously at 105° C. After 10 min, deionized water (100 μL) is added with stirring, followed by additional aliquots at 20 min (50 μL), 30 min (75 μL), and 60 min (125 μL). The insoluble materials can be removed by centrifugation, rinsed twice with water (200 μL), and dried. The liquid products (2.046 g) can be analyzed by HPLC (2.0 mg/g glucose, 42% yield; 2.3 mg/g xylose, 71% yield).

Optionally, the acidolysis can be performed a second time. The brown solids from the first hydrolysis are heated with EMIM Cl (306 mg) at 105° C. for 4.5 h. To this mixture is added aqueous HCl (1.66 M, 14.5 μL, equivalent to 2.5 mg conc. HCl), and the reaction mixture is vigorously stirred at 105° C. After 10 min, deionized water (50 μL) is added with stirring, followed by an additional 25 μL water at 20 min, 67.5 μL water at 30 min, and 70 μL water at 60 min. The liquid products (770 mg) can be analyzed by HPLC (3.56 mg/g glucose, 28% yield; 0.7 mg/g xylose, 8% yield).

For the two-step process described above, the overall yield of glucose is 70% and the overall yield of xylose is 79%. Optionally, water can be added to the IL or ILA phase wherein the proportion of water in the IL or ILA phase does not exceed about 60% by weight.

### Example 2

### Acid Catalysis of Switchgrass in Ionic Liquids with Extraction of Fermentable Sugars

Ionic liquids (ILs) are of interest as a new solvent for the pretreatment of biomass. The realization of an economically-viable pretreatment technology employing ionic liquids requires the near complete conversion of biomass into its component sugars and recycle of the ionic liquid solvent. The IL 1-butyl-3-methyl imidazolium chloride ([C4mim][Cl]) has been shown to dissolve biomass and subsequently accelerate the rates of enzymatic hydrolysis of holocellulose that is precipitated from biomass dissolved in IL solvents into monomeric sugars. [C4mim][Cl] has also been used to produce sugars in situ using mineral acids such as HCl. Recently, we reported the use of boronic acids to recover sugars from an aqueous-IL phase into an organic phase. The present work employs HCl catalysis of switchgrass dissolved in 1-ethyl-3-methyl imidazolium chloride [C2mim][Cl], and uses naphthalene-2-boronic acid (N2B) in solution with hexane and Aliquat 336™ to recovery more than 90% of the total sugars produced by the acid hydrolysis. This IL treatment process provides an alternative route to the production of monomeric sugars from biomass that may eliminate the need for saccharolytic enzymes, and could potentially provide a scalable and economical route to the production of biofuels and other biomass derived products.

### INTRODUCTION

Ionic liquids continue to gain interest as a novel class of solvents for a variety of industrial chemical processes. These environmentally-benign solvents have shown great promise for pretreating biomass by disrupting the highly structured intra-molecular H-bonding matrix present in plant cell walls. After dissolution, the addition of an anti-solvent, such as water, results in the precipitation of most of the cellulose and some of the hemicellulose components of the biomass. The resulting polysaccharides can be hydrolyzed using saccharolytic enzymes, liberating monomeric sugars that are converted to fuels by microbial fermentation. However, there are several processing challenges surrounding current ionic liquid based pretreatment techniques. First, saccharolytic enzymes costs represent up to 25% of the total material costs for biomass-to-biofuel processing and require processing times up to 72 hours for the complete conversion of pretreated polysaccharides to individual sugar subunits. Second, ionic liquids reduce the activity of cellulolytic enzymes. Although there is encouraging work in the development of IL-tolerant enzymes, successive washing of the pretreated biomass is required to remove residual ionic liquids. Therefore, large amounts of water are required, making complete recovery and recycle of the ILs difficult. Although a commercial IL-based biomass deconstruction process is not yet realized, the use of saccharolytic enzymes may result in high costs associated with IL recovery and purification.

Coupling mineral acid catalysis of biomass dissolved in ILs and sugar extraction technology provides an alternative route to monomeric sugar production that may eliminate the need for saccharolytic enzymes. The use of mineral acids in the presence of ILs for the catalytic hydrolysis of lignocellulosic material offers an alternative to enzymatic release of sugar monomers. Raines and Binder developed an effective method for the acid-catalyzed release of sugars from corn stover, showing the gradual addition of water to [C2mim][Cl] treated biomass solubilizes the cellulose polysaccharides and stabilizes the glucose oligomers, providing easily-recovered sugars. However, in any proposed IL-based treatment technology, the near complete conversion of biomass, efficient sugar recovery and the near complete recycle of the ionic liquid solvent is required for an economically-viable scheme. We propose that the liquid-liquid extraction method using the affinity of boronic acid for C₅ and C₆ sugars in the presence of ILs could provide a route to address these two critical processing challenges. We have shown in pure IL solvents and IL-water mixtures the recovery of over 80% of soluble sugars can be achieved in less than 3 hours using boronic acid extraction. This approach was combined with that developed by Raines and Binder for holosaccharide hydrolysis, and is the focus of this paper. Combining acidolysis of biomass with boronic acid extraction of sugars may provide a fast, scalable and economical route to the production of biofuels or other biomass-derived products.

### MATERIALS AND EXPERIMENTAL METHODS

### Acidolysis of Switchgrass in Ionic Liquid

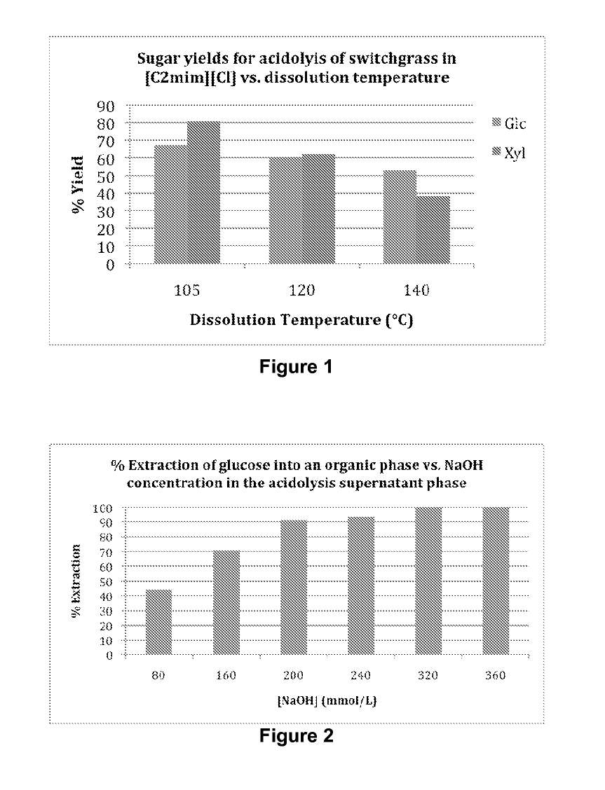

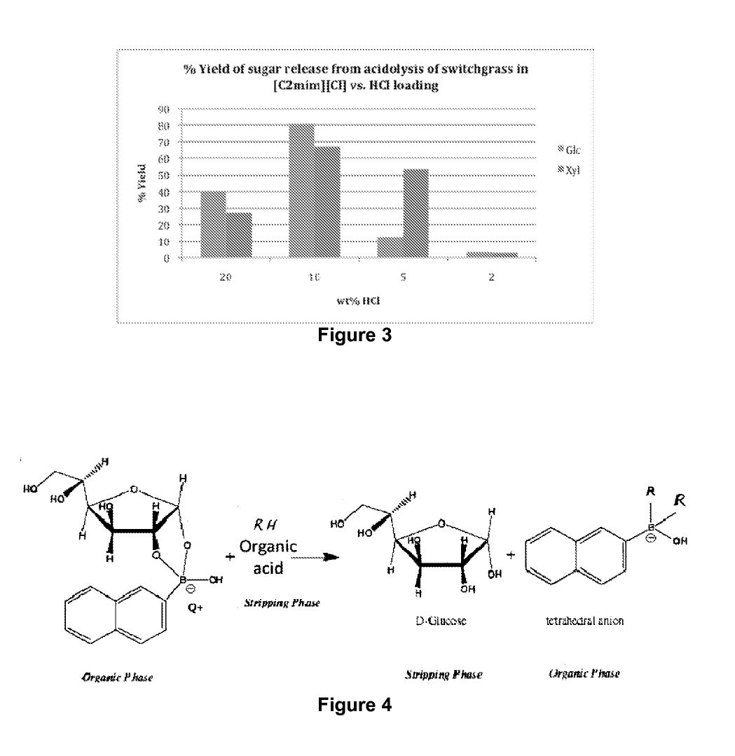

The acid catalyzed hydrolysis of switchgrass was carried out based on a variant of the method established by Raines and Binder [PNAS 2010]. A sample of 0.1 g (5 wt %) of switchgrass (MPV2: 33.13 dry wt % glucan and 16.43 dry wt % xylan (<60 mesh (422-251 um)) was added to 2 g of [C2mim][Cl] (BASF Quality>=95% by Aldrich) in 30 ml glass vials (Kimble HS No. 45600-30). Prior to the addition of the acid catalyst, the solution was heated and mixed using a Thermo Scientific STEM mixer (cat no. PS 80068) for 6 hours at 105° C. and 800 rpm. This 6 hr premixing is referred to as the dissolution time in FIG. 1. The dissolution temperature was also tested for optimizing sugar release. This was conducted using the same protocol but premixing at 120° C. and 140° C. for 5 hrs and then allowing the temperature to equilibrate to the reaction temperature (105° C.) for the last hour (6 hr total for dissolution time) prior to acid catalyst addition. At the end of the dissolution period the acid catalyst was added (t=0) in an aqueous solution. At t=0, the hydrochloric acid (4M) loading was 10 wt % of the biomass and the water loading was 5 wt % of the total reaction mass. The 4M HCl solution was prepared by adding 66.67 ml of 6M HCl (VWR Cat no. 3204-1) and adjusting the final volume to 100 ml with water. An aliquot of 68.6 mg of 4M HCl was mixed with 52.6 mg of water (5 wt %) and then added to the IL-switchgrass dissolution solution to initiate the hydrolysis reaction. Then water was gradually added to the reaction mixture as follows: at 10 minutes from t=0, 482 μL of water (20 wt %) were added, at 20 minutes 180 μL of water (25 wt %) were added, 30 minutes (344 μL, 33 wt %) and 60 minutes (566 μL, 43 wt %). After 60 minutes no water was added. At 2.5 hours, the reaction mixture was diluted with 3 ml of water, centrifuged to separate the insoluble material, and aliquots of the soluble phase were analyzed for sugar characterization and quantification. Following the acidolyisis method described above, the HCl concentration was varied (2, 5, 10, 20 wt % HCl, FIG. 3), keeping the [C2mim][Cl] mass and initial switchgrass mass constant. Additionally, the amount of water added at 2.5 hours to dilute the reaction was tested. Two separate hydrolysis trials were conducted using the same protocol. At 2.5 hours, in the first reaction 1.5 ml of water was added and in the second no water was added for dilution (compared to the 3 ml described by Raines and Binder). The two vials were then centrifuged to separate the insoluble material, and aliquots of the soluble phase were analyzed for sugar characterization and quantification. The sugar release yields were molar yields based on HPAEC analysis and relative to the sugar subunits contained in the switchgrass starting material.

### Analytical Methods

Sugar concentrations were measured using High Performance Anion Exchange Chromatography with Pulsed Amperometric Detection (HPAEC-PAD) on a Dionex DX3000 equipped with a Dionex Carbopac PA-20 analytical column (3×150 mm) and a Carbopac PA-20 guard column (3×30 mm) (Dionex, Sunnyvale, Calif., USA). Eluent flow rate was 0.4 ml/min and the temperature was 30° C. A gradient consisting of a 14 min elution with 11 mM NaOH followed by a 11 min gradient to 450 mM NaOH for 5 min, then a return to the original NaOH concentration of 11 mM for 7 min prior to the next injection was employed. Hydroxy-methylfurfural (HMF) was also analyzed using the HPAEC. A standard calibration curve was produced using know quantities and [HMF] was monitored in the final acidolysis supernatant.

### Extraction of Sugars

The extraction of sugars from the IL-water hydrolysis supernatant to an upper organic phase was carried out using the boronic acid liquid-liquid extraction method for C₅ and C₆ sugar monomers [Brennan et. al., “Recovery of Sugars from Ionic Liquid Biomass Liquor by Solvent Extraction”, BioEnergy Research. 2010 3(2), p. 123]. Following the acidolysis of switchgrass, the hydrolysis IL-supernatant was spiked with 10 μL of 10M NaOH (200 mM [NaOH] in solution), vortexed and pH adjusted to pH 11-12 before extraction. In addition, the amount of NaOH added (4-18 μL equivalent to 80-360 mM NaOH) was tested for optimal extraction efficiency (FIG. 2) and the additional volume can be treated as negligible relative to the reaction volume (0.5 ml). Applying our previous works extraction technique [Brennan et. al. BioEnergy 2010], 0.5 ml of an n-hexane:octanol (85:15 v/v) organic phase containing 70 mM naphthalene-2-boronic acid (97% purity by Fischer Scientific) was mixed in equal volume (0.5 ml) with the hydrolysis IL-supernatant for 2 hours at 70° C. and 1400 rpm (VWR thermomixer). The final mixture (1 ml) was then centrifuged (13000 rpm for 30 sec) for phase separation and the hydrolysis IL-supernatant phase was analyzed for final sugar concentration. As described in our previous work [Brennan et. al. BioEnergy 2010], the initial and final sugar concentrations of the hydrolysis supernatant were used to calculate the % extraction of soluble sugars from the IL-supernatant phase into the upper organic phase.

### Recovery of Sugars

The recovery of sugars from the loaded organic phase into an aqueous dilute acid phase again followed our previous stripping protocol [Brennan et. al. BioEnergy 2010] with one addition. The loaded organic phase was premixed with 400 μL of hexanoic acid (Aldrich Chemicals) for 0.5 hr at 70° C. and 1400 rpm. This organic phase (total final volume 0.9 ml (0.5 ml sugar-loaded organic+0.4 ml hexanoic acid)) was then mixed with 0.5 ml of aqueous 0.5M acetic acid (HAc) for 0.5 hr at 70° C. and 1400 rpm. In addition to using 0.5M HAc, a more concentrated acid solution was tested for sugar release (8M HAc). The final mixture was then centrifuged (13000 rpm for 30 sec) for phase separation and the lower aqueous phase was analyzed for final sugar concentration. As described in our previous work [Brennan et. al. BioEnergy 2010], the initial and final sugar concentrations in the organic phase were used to calculate the % recovery of sugars from the upper sugar-loaded organic phase into the lower aqueous dilute acid phase.

### RESULTS AND DISCUSSION

### Acidolysis of Switchgrass

The optimal acid loading was determined as 10 wt % HCl (FIG. 3) for a 5 wt % switchgrass loading in [C2mim][Cl]. Using twice this acid loading (20 wt %) resulted in roughly half the sugar release compared to 10 wt % HCl (27% yield and 67% yield for glucose in FIG. 3). For a single batch run, a 25% higher yield based on glucose (67% yield) and 10% higher yield for xylose (81% yield) release was observed during the acidolysis reaction compared to the reported literature values (42% glucose and 71% xylose yields from corn stover [Raines and Binder, PNAS 2010], Table 1). The HMF concentration in the IL-supernatant was 0.6 μM and was treated as negligible. Testing the amount of water required for dilution at the end of the hydrolysis reaction at 2.5 hours produced encouraging results. We report that at 2.5 hrs, the addition of no water and 1.5 ml (50% less than the literature protocol of 3 ml) resulted in the same sugar yields for both glucose and xylose (Table 1). The development of an economically viable IL pretreatment process is highly dependent on the ability to recycle and regenerate the ionic liquid for closed loop processing. Reducing water requirements while maintaining sugar recovery in an IL-based pretreatment process could significantly benefit the scalability of ionic liquid use as a pretreatment for lignocellulosic biomass.

Table 1. Switchgrass (5 wt %) was reacted with [C2mim][Cl] and 10 wt % (to biomass) HCl at 105° C. for 2.5 hours with gradual water addition. At the end of the reaction time of 2.5 hrs, the reaction mixture was diluted. The water content is relative to the entire reaction mass after the dilution volume was added. Sugar yields are molar yields analyzed using HPAEC and based on sugar units contained in the switchgrass feedstock. The standard deviation (SD) is based on triplicate trials for 3 ml dilution reactions only.

TABLE 1

| Dilution | Water content after | Glucose % | Xylose % |
|---|---|---|---|
| Volume (ml) | dilution (wt %) | Yield, (SD) | Yield, (SD) |
| 3 | 69 | 67, (5) | 81, (3) |
| 1.5 | 60 | 64 | 81 |
| 0 | 44 | 64 | 79 |

Increasing the dissolution temperature from 105° C. prior to acidolysis initiation had little effect on sugar release (for glucose at 105, 120, 140° C.: 67, 60, 53% yield). In FIG. 1, the sugar yields are reduced with increasing dissolution temperatures. This affect could be due to the formation of furfurals (HMF), other unwanted byproducts (which were not tested) or structural changes to the cellulose or hemicellulose that may have inhibited the acid catalyst efficiency during the reaction.

### Sugar Extraction and Recovery

The effective extraction of glucose and xylose monomers (91 and 97% extraction) from the acidolysis supernatant provides an excellent processing opportunity. As opposed to potentially expensive chromatography techniques required to recover both the sugar monomers and the ILs, liquid-liquid extraction using the ability of boronic acid to chelate with soluble sugars in an IL-water mixture, followed by phase separation, offers a practical alternative to sugar recovery and IL recycle. The extraction efficiency of the boronic acids is dependent on pH. In high pH environments (above pH 9), the boronic acids undergo a structural change from a trigonal to tetrahedral form, which augments the formation of the brononic acid-sugar ester complex. Therefore, the NaOH concentration in the IL-supernatant phase was tested for extraction efficiency (FIG. 2) and an optimal NaOH concentration was chosen (200 mM) for subsequent extraction trials.

Although the results we report are encouraging only for glucose recovery (67% glucose recovery, <2% xylose recovery), more work is necessary to fully understand the mechanism of boronic ester breakdown in the organic environment and the optimal conditions necessary to facilitate higher sugar recovery. We believe that the sugar release from the boronic acid-sugar ester complex could be highly dependent on the [OH⁻] activity within the loaded organic phase. The most successful sugar recovery came from the addition of an organic acid (hexanoic acid) to the loaded hexane-boronic acid-sugar phase prior to contact/mixing with the aqueous acid phase (67% glucose recovery with hexanoic acid premix, 28% recovery without, data not shown). This result could indicate that the hexanoic acid assisted in the sequestration of [OH⁻] ions allowing for the protonation of the oxygen atoms in the boronic acid ester complex and thus facilitating esterification and sugar release (proposed reaction in FIG. 4). Although future work would be necessary to delineate the mechanism of sugar release, our reported results offer an excellent starting point for the development of liquid-liquid extraction and carbohydrate recovery technology in ionic liquids.

### CONCLUSION

The consolidation of acid catalysis and boronic acid extraction of fermentable sugars in ionic liquids has been shown to be an effective technology for the pretreatment of lignocellulosic biomass. This IL-based process offers an alternative pretreatment route for the release of monomeric sugars that could potentially eliminate the use of enzymes for biofuel production. The selective extraction of monomeric sugars also enables the recovery and recycle of ionic liquids, as well as the fractionation and recovery of lignin after sugar recovery in a separate liquid-liquid extraction process. The coupling of these two technologies could potentially provide a scalable and economically viable processing technology for the production of biofuels or other biomass based products.

Table 2. The recovery of sugars from 10 wt % HCl acidolysis of switchgrass. The % extraction is the amount of sugar removed from the IL acidolysis supernatant on a molar basis into the organic phase containing 70 mM naphthalene-2-boronic acid. The % recovery is the amount of sugars on a molar basis released from the loaded organic phase into an aqueous solution of acetic acid (AcOH). Results for two different [AcOH] are shown.

TABLE 2

| | | | Recovery % (8M, |
|---|---|---|---|
| Sugar | Acidolysis % Yield | Extraction % | 0.5M HAc) |
| Glucose | 67 | 91 | 67, 55 |
| Xylose | 81 | 97 | <2 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of hydrolyzing a cellulose, hemicellulose, or ligno-cellulose comprising:
(a) contacting (i) an ionic liquid (IL) or ionic liquid-aqueous (ILA) phase comprising cellulose, hemicellulose, or ligno-cellulose, or a mixture thereof, and (ii) an acid, such that the cellulose, hemicellulose, or ligno-cellulose is hydrolyzed into a sugar.

2. The method of claim 1, further comprising: (b) adding water to the IL or ILA phase, wherein the proportion of water in the IL or ILA phase does not exceed about 60% by weight.

3. The method of claim 1, wherein the acid has a pH equal to or lower than about the pH of 4M hydrochloric acid.

4. The method of claim 3, wherein the acid is HCl.

5. The method of claim 1, further comprising:
(c) contacting the IL or ILA phase and an organic phase optionally comprising an organic acid, wherein the solution comprises an ionic liquid, a sugar and a boronic acid;
(d) contacting the sugar with the boronic acid to form a sugar-boronic acid complex, and
(e) separating the organic phase and the aqueous phase, wherein the organic phase contains the sugar-boronic acid complex.

6. The method of claim 5, further comprising: (f) adding a stripping agent to the organic phase.

7. The method of claim 6, further comprising: (g) separating the sugar from the organic phase.

8. A composition comprising a solution comprising:
(a) an ionic liquid (IL) or ionic liquid-aqueous (ILA) phase and
(b) an organic phase, wherein the solution comprises a sugar, a boronic acid, and an organic acid.

9. The composition of claim 8, wherein the organic acid is a carboxylic acid.

10. The composition of claim 9, wherein the organic acid is an alkanoic acid.

11. A method of removing a sugar from a solution, comprising:
(a) providing a solution comprising (i) an ionic liquid (IL) or ionic liquid-aqueous (ILA) phase and (ii) an organic phase comprising an organic acid, wherein the solution comprises an IL, a sugar and a boronic acid;
(b) contacting the sugar with the boronic acid to form a sugar-boronic acid complex; and
(c) separating the organic phase and the aqueous phase, wherein the organic phase contains the sugar-boronic acid complex.

12. The method of claim 11, further comprising: (d) separating the sugar from the organic phase.

13. The method of claim 11, wherein the (a) providing step comprises contacting (i) an IL or ILA solution and (ii) an organic solution comprising an organic acid, whereby the pH of the IL or ILA solution is lowered.

14. The method of claim 13, wherein the IL or ILA solution comprises the IL, the sugar, and the boronic acid.

15. The method of claim 12, wherein the (d) separating step comprises: (i) contacting the organic phase and a second aqueous phase, whereby the sugar-boronic acid complex dissociates into the sugar and the boronic acid and the sugar moves into the second aqueous phase.

16. The method of claim 15, wherein the (d) separating step further comprises: (ii) separating the organic phase and the second aqueous phase.

17. The method of claim 15, wherein the second aqueous phase comprises an alkanoic acid.

18. The method of claim xx further comprising: (e) culturing a cell using the sugar obtained from the (d) separating step.

19. The method of claim 18, wherein the organic phase comprises a biomass comprising the sugar.

20. The method of claim 19, wherein the biomass is a cellulose biomass, hemicellulose biomass, ligno-cellulose biomass, or a mixture thereof.
